(19) United States
(12) Patent Application Publication
(10) Pub. No.: US 2005/0070565 A1
(43) Pub. Date: Mar. 31, 2005

(54) Chelerythrine, analogs thereof and their use in the treatment of bipolar disorder and other cognitive disorders

(76) Inventors: Amy F.T. Arnsten, Bethany, CT (US); Shari G. Birnbaum, Houston, TX (US)

Correspondence Address: COLEMAN SUDOL SAPONE, P.C., 714 Colorado Avenue, Bridgeport, CT, 06605-1601, US

(21) Appl. No.: 10/672,626

(22) Filed: Sep. 26, 2003

(51) Int. Cl. A61K 31/4741; A61K 31/473

(52) U.S. Cl. 514/283; 514/284

(57) ABSTRACT

The present invention relates to the use of chelerythrine and chelerythrine analogs in pharmaceutical compositions for the treatment of prefrontal cortical cognitive disorders, including bipolar disorder, among others.

### FIELD OF THE INVENTION

The present invention relates to the use of chelerythrine and chelerythrine analogs in pharmaceutical compositions for the treatment of neuropsychiatric disorders that involve dysfunction of the prefrontal cortex, including bipolar disorder, among others.

### BACKGROUND OF THE INVENTION

Converging evidence indicates that overactivity of the intracellular signaling enzyme, protein kinase C, gives rise to manic symptomology in bipolar disorder. There are both higher levels of protein kinase C, and increased activity of protein kinase C in the cortex of manic patients, and all effective anti-manic agents have protein kinase C blocking activity (reviewed in Manji and Lenox, 1999). For example, the widely used antimanic, nonselective agent lithium reduces protein kinase C activity by blocking inositol phosphate phosphatase and decreasing the availability of precursor (myoinositol) in the phosphotidylinositol cascade (Sun et al., 1992). Indeed, proton magnetic resonance spectroscopy studies of bipolar patients have shown that lithium treatment significantly reduces myoinositol levels in the right prefrontal cortex, a brain region strongly linked to manic symptoms (see below). A recent “proof of concept” study showed that tamoxifen, an anti-estrogen compound with protein kinase C-blocking activity at higher concentrations, ameliorated manic symptoms when administered at higher doses (Bebchuk et al., 2000), suggesting that protein kinase C blockade is indeed therapeutic in bipolar disorder.

The prefrontal cortex regulates human behavior using working memory, inhibiting inappropriate impulses and reducing distractibility (Goldman-Rakic, 1996; Robbins, 1996). In humans, the prefrontal cortex in the right hemisphere is particularly important for inhibiting inappropriate impulses, and reduced size of this cortex correlates with disinhibited behavior (Casey et al., 1997). Thus, the profound disinhibition during manic episodes typifies prefrontal cortical dysfunction. This has been confirmed by imaging studies: There is a reduction in size of the prefrontal cortex in patients with bipolar disorder (Drevets et al., 1997), and the medial/orbital portion of the prefrontal cortex on the right side is markedly underactive in bipolar patients during the manic state (Blumberg et al., 1999).

Manic episodes in bipolar patients can be precipitated by exposure to stress (Hammen and Gitlin, 1997). Either environmental stressors (such as very loud noise, e.g., greater than 95 dB) or pharmacological stress (the partial inverse benzodiazepine agonist, FG7142) can impair prefrontal cortical function in monkeys and rats, while having no effect on cognitive performance unrelated to the prefrontal cortex (Arnsten, 1998; Arnsten and Goldman-Rakic, 1998; Murphy et al., 1996). Similarly, humans exposed to stressful levels of noise demonstrated deficits in prefrontal cortical function (Hartley and Adams, 1974), particularly when the subject experienced no control over the stressor (Glass et al., 1971). High levels of dopamine and norepinephrine are released into the prefrontal cortex during stress exposure, and these excessive levels of catecholamines impair prefrontal cortical function by stimulating D1 dopamine receptors and alpha-1 adrenoceptors, respectively (reviewed in Arnsten, 2000).

Overstimulation of D1 receptors impairs prefrontal cortical function via excessive activation of the protein kinase A signalling pathway (Taylor et al., 1999), while overstimulation of alpha-1 receptors impairs cognitive function via excessive activation of the protein kinase C signalling pathway (FIG. 1, and see below). As manic patients are especially susceptible to overactivity of protein kinase C, this would lead to dysfunction of the prefrontal cortex, and symptoms of prefrontal cortex dysfunction such as impulsivity, distractibility, and poor judgement, which are key features of mania.

The deficits in prefrontal cortical function that occur during stress can be mimicked by stimulating the prefrontal cortex with a nonadrenergic alpha-1 agonist. Thus, systemic administration of an alpha-1 agonist that crosses the blood brain barrier (Arnsten and Jentsch, 1997), or direct infusion of an alpha-1 agonist into the prefrontal cortex (Arnsten et al., 1999; Mao et al., 1999) impairs working memory performance in monkeys and rats. This impairment can be reversed by either systemic administration or local application of an alpha-1 adrenoceptor antagonist (ibid; FIG. 2). Direct infusion of an alpha-1 adrenoceptor antagonist into the PFC also prevents stress-induced cognitive deficits, thus demonstrating the importance of this pathway in the stress response (Birnbaum et al., 1999, FIG. 3).

Alpha-1 adrenoceptors are most commonly linked by Gq to the phosphotidyl inositol cascade and activation of protein kinase C (Duman and Nestler, 1995; FIG. 1). Recent experiments indicate that both stress and alpha-1 agonists impair prefrontal cortical function through activation of this intracellular signalling pathway. The impairment induced by alpha-1 adrenergic agonist infusion into the prefrontal cortex is reversed by a dose regimen of lithium treatment known to suppress phosphotidyl inositol turnover (Arnsten et al., 1999; FIG. 4). Similarly, oral administration of a clinically relevant dose of lithium to monkeys prevents prefrontal cortical cognitive impairment due to the alpha-1 adrenergic agonist, cirazoline (FIG. 5).

Accordingly, the need exists for selective methods of treating impaired prefrontal cortical function associated with uncontrollable stress. Similarly, the need exists for selective methods of protecting cognitive performance from stress exposure.

### SUMMARY OF THE INVENTION

Applicants have discovered in animal studies that exposure to uncontrollable stress impairs prefrontal cortical function via activation of protein kinase C, and that administration of chelerythrine or a chelerythrine analog in accordance with the invention inhibits harmful protein kinase C activation. Accordingly, the invention provides compositions and methods useful in treating a subject suffering from a CNS disorder, particularly a CNS disorder associated with impaired prefrontal cortical function related to activation of protein kinase C due to exposure to uncontrollable stress. In particular, the invention provides compositions and methods that treat a subject suffering from such disorders by administering to the subject an effective amount of the selective protein kinase C inhibitor chelerythrine or a chelerythrine analog as defined hereinafter.

Additionally, the invention provides a method of protecting a subject's cognitive performance from alpha-1 receptor stimulation or stress exposure by administering to the subject an effective amount of the selective protein kinase C inhibitor chelerythrine or a chelerythrine analog.

CNS disorders that may be treated by compositions and methods of the claimed invention include bipolar disorder, major depressive disorder, schizophrenia, post-traumatic stress disorder, anxiety disorders, attention deficit hyperactivity disorder, and Alzheimer's Disease (behavioral symptoms).

In one embodiment, the invention relates to a method comprising treating a subject suffering from a disorder associated with impaired prefrontal cortical function associated with activation of protein kinase C by administering to the subject a pharmaceutical composition comprising an effective amount of chelerythrine, which has the following formula: 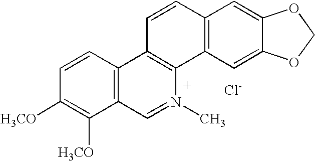
and stereoisomers, pharmaceutically acceptable salts, solvates, and polyimorphs thereof.

In another embodiment, the invention relates to a method comprising treating a subject suffering from a disorder associated with impaired prefrontal cortical function associated with activation of protein kinase C by administering to the subject a pharmaceutical composition comprising an effective amount of chelerythrine analog, which is defined herein as a compounds of formulae (I) or (II) 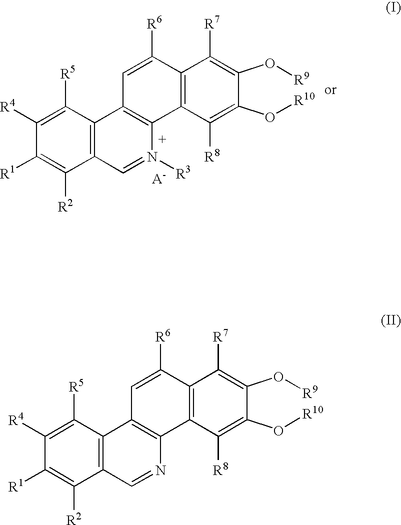
wherein:
R¹ and R² are independently selected from H, C₁-C₃ alkyl, F, Cl, Br, I, OH, O(C₁-C₆ alkyl), O—C(═O)—(C₁-C₆)alkyl, C(═O)—O—(C₁-C₆)alkyl, more preferably O-alkyl, even more preferably, OCH₃;
R³ is H or a C₁-C₆ alkyl group, preferably a methyl or ethyl, most preferably methyl;
R⁴, R⁵, R⁶, R⁷ and R⁸ are independently selected from H, C₁-C₆ alkyl, F, Cl, Br, I, OH, —(CH₂)ₙO(C₁-C₆ alkyl), —(CH₂)ₙO—C(═O)—(C₁-C₆)alkyl, —(CH₂)ₙC(═O)—O—(C₁-C₆)alkyl;
R⁹ and R¹⁰ are independently H, C₁-C₆ alkyl, preferably C₁-C₃ alkyl or together form a —(CH₂)ₘ— group to produce a 5-7-membered ring;
n is from 0 to 5;
m is from 1 to 3;
and A- is a pharmaceutically acceptable anion of a pharmaceutical salt, which forms a salt with the quaternized amine group, and can be F—, Cl—, Br—, I—, sulfate, citrate, tartrate, phosphate, etc, or a stereoisomer, pharmaceutically acceptable salt, solvate, and polymorph thereof.

In preferred embodiments, the compositions and methods of the invention use compounds of formulae (I)-(II) that represent minor modifications of chelerythrine.

Compounds useful in the invention may be synthesized by methods readily available in the art. For example, derivation of commercially available isoquinoline analogs may readily form the third and fourth ring structure (and even the fifth ring structure, where applicable) with an appropriately derivitized benzaldehyde to be condensed onto the isoquinoline analog.

These and other aspects of the invention are described further in the following detailed description.

### BRIEF DESCRIPTION OF THE FIGURES

FIG. 1 provides a schematic depiction of the phosphotidyl inositol protein kinase C (PI/PKC) intracellular signaling cascade and its stimulation by α-1 adrenergic receptor stimulation.

FIG. 2 illustrates that infusion of the alpha-1 agonist, phenylephrine, into rat prefrontal cortex impairs working memory, and that this impairment is prevented by co-infusion of the alpha-1 antagonist, urapidil.

FIG. 3 illustrates that stress exposure impairs working memory and induces cognitive deficits in rats, and that this impairment is prevented by the infusion of the alpha-1 antagonist, urapidil, into the prefrontal cortex.

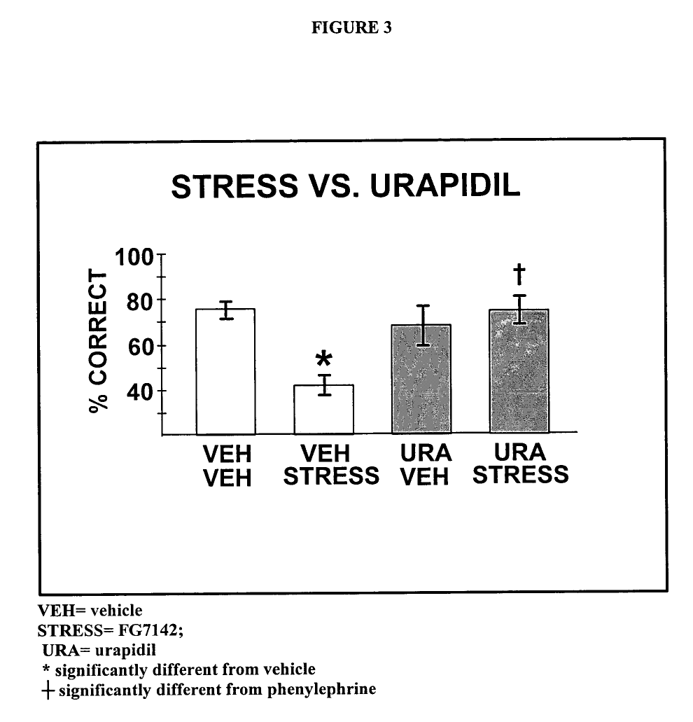

FIG. 4 shows that a dose of lithium known to suppress phosphotidyl inositol turnover reverses the impairing effects of an alpha-1 agonist infused into the prefrontal cortex or rats.

FIG. 5 illustrates that pretreatment with a clinically relevant dose of lithium (5-7.5 mg/kg p.o.) reverses the deficits induced by systemic administration of the alpha-1 agonist cirazoline to rhesus monkeys.

FIG. 6 shows that impairment in working memory performance caused by administration of phenylephrine is significantly blocked by co-infusion of chelerythrine.

FIG. 7 illustrates that co-infusion of chelerythrine (0.3 μg/0.5 μl) into the rat PFC significantly reversed the detrimental effects of stress exposure.

FIG. 8 illustrates that systemic (s.c.) administration of chelerythrine significantly reduces the cognitive deficits induced by stress exposure in rats.

FIG. 9 illustrates that oral chelerythrine (0.03-0.3 mg/kg, p.o.) prevents stress induced prefrontal cortical dysfunction in rhesus monkeys.

FIG. 10 illustrates a summary whereby treatment with chelerythrine reverses the detrimental effects of: A. infusions of the protein kinase C activator, PMA, into the rat prefrontal cortex; B. infusions of the alpha-1 agonist, phenylephrine, into the rat prefrontal cortex; and C. administration of the alpha-1 agonist, sirazoline, in rhesus monkeys. This figure shows the summary of effect of PKC activation (direct or indirect) on working memory. In A., direct activation of PKC by infusion of the phorbol ester, PMA, directly into the prefrontal cortex significantly impaired delayed alternation performance compared to vehicle treatment in rats (ANOVA-R; vehicle+vehicle vs. PMA+vehicle: * F_{1,8}=26.45, p=0.001). A dose of PMA was found for each individual animal that impaired delayed alternation testing (range: 0.05 to 5 pg/0.5 μl). The PMA-induced working memory deficit was reversed by co-infusion of the PKC inhibitor, chelerythrine (CHEL, 0.3 μg/0.5 μl; PMA+vehicle vs. PMA+chelerythrine: † F_{1,8}=46.50, p<0.001). Chelerythrine had no effect on its own. B. Indirect activation of PKC by infusion of the α-1 adrenergic receptor agonist, phenylephrine (PE, 0.1 μg/0.5 μl) directly into the prefrontal cortex significantly impaired delayed alternation performance compared to vehicle treatment in rats (vehicle+vehicle vs. phenylephrine+vehicle: * F_{1,8}=11.10, p=0.01). The phenylephrine-induced working memory deficit was reversed by co-infusion of chelerythrine (phenylephrine+vehicle vs. phenylephrine+chelerythrine: † F_{1,8}=8.01, p<0.022). Chelerythrine had no effect on its own. C. In monkeys, indirect activation of PKC by systemic administration of the α-1 adrenergic receptor agonist, cirazoline (CIRAZ) significantly impaired delayed response performance compared to vehicle treatment (vehicle+vehicle vs. cirazoline+vehicle: * F_{1,4}=26.74, p=0.007). A dose of cirazoline (range: 0.001 to 10 μg/kg) was determined for each animal that reliably impaired delayed response testing. The cirazoline-induced working memory deficit was reversed by pretreatment with chelerythrine (0.03 mg/kg; cirazoline+vehicle vs. cirazoline+chelerythrine: † F_{1,4}=11.10, p=0.008). Chelerythrine had no effect on its own.

FIG. 11 illustrates a summary whereby treatment with chelerythrine reverses the detrimental effects of: A. stress in rats; or B. stress in monkeys. C. illustrates that infusion of chelerythrine into the rat prefrontal cortex did not reverse stress-induced freezing. This figure shows the effect of PKC inhibition on the stress-induced cognitive impairment in rats and monkeys. In A, the anxiogenic stressor, FG7142 (range: 10 to 20 mg/kg), impaired delayed alternation performance compared to vehicle treatment in rats (vehicle+vehicle vs. FG7142+vehicle: * ANOVA-R, F_{1,10}=25.095, p=0.001). The FG7142-induced cognitive deficit was reversed by infusion of the PKC inhibitor, chelerythrine, into the prefrontal cortex 15 min prior to testing (0.3 μg/0.5 μl; FG7142+vehicle vs. FG7142+chelerythrine: † F_{1,10}=10.170, p=0.010). In B, in monkeys, injection of FG7142 (range: 0.2 to 2.0 mg/kg) significantly impaired delayed response performance compared to vehicle treatment (vehicle+vehicle vs. FG7142+vehicle: * F_{1,5}=20.69, p=0.006). The FG7142-induced cognitive deficit was reversed by pretreatment with the PKC inhibitor, chelerythrine (0.03 mg/kg; FG7142+vehicle vs. FG7142+chelerythrine: † F_{1,4}=21.23, p=0.006). In C, following FG7142 administration, rats often exhibit stress-related behaviors such as freezing and grooming. These behaviors do not depend on prefrontal cortical function, but can increase time to complete each trial (average response time for each trial for vehicle+vehicle vs. FG7142+vehicle: * F_{1,10}=12.264, p=0.006). This increased response time induced by FG7142 was not blocked by chelerythrine (FG7142+vehicle vs. FG7142+chelerythrine: F_{1,10}=0.283, p=0.606; vehicle+vehicle vs. FG7142+chelerythrine: * F_{1,10}=14.502, p=0.003).

### DETAILED DESCRIPTION OF THE INVENTION

As used herein, the following terms have the following respective meanings. Other terms that are used to describe the present invention have the same definitions as those generally used by those skilled in the art. Specific examples recited in any definition are not intended to be limiting in any way.

“Hydrocarbon” refers to a substituted or unsubstituted organic compound.

“Acetal” refers to a compound in which two ether oxygens are bound to the same carbon. A “ketal” is an acetal derived from a ketone.

“Acyl” means a compound of the formula RCO, where R is aliphatic (characterized by a straight chain of carbon atoms), alicyclic (a saturated hydrocarbon containing at least one ring), or aromatic.

“Acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein

“Alkyl” refers to a fully saturated monovalent hydrocarbon radical containing carbon and hydrogen which may be a straight chain, branched, or cyclic. Examples of alkyl groups are methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl. “Cycloalkyl” groups refer to cyclic alkyl groups such as cyclopropyl, cyclobutyl, cyclopentyl and cyclohexyl. C₁-C₇ alkyl groups are preferably used in the present invention.

“Substituted alkyl” refers to alkyls as just described which include one or more functional groups such an alkyl containing from 1 to 6 carbon atoms, preferably a lower alkyl containing 1-3 carbon atoms, aryl, substituted aryl, acyl, halogen (i.e., alkyl halos, e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like. The term “substituted cycloalkyl” has essentially the same definition as and is subsurned under the term “substituted alkyl” for purposes of describing the present invention.

“Amine” refers to aliphatic amines, aromatic amines (e.g., aniline), saturated heterocyclic amines (e.g., piperidine), and substituted derivatives such as an alkly morpoline. “Amine” as used herein includes nitrogen-containing aromatic heterocyclic compounds such as pyridine or purine.

“Aralkyl” refers to an alkyl group with an aryl substituent, and the term “aralkylene” refers to an alkenyl group with an aryl substituent. The term “alkaryl” refers to an aryl group that has an alkyl substituent, and the term “alkarylene” refers to an arylene group with an alkyl substituent. The term “arylene” refers to the diradical derived from aryl (including substituted aryl) as exemplified by 1,2-phenylene, 1,3-phenylene, 1,4-phenylene, 1,2-naphthylene and the like.

“Alkenyl” refers to a branched or unbranched hydrocarbon group typically although not necessarily containing 2 to about 24 carbon atoms and at least one double bond, such as ethenyl, n-propenyl, isopropenyl, n-butenyl, isobutenyl, octenyl, decenyl, and the like. Generally, although again not necessarily, alkenyl groups herein contain 2 to about 12 carbon atoms. The term “lower alkenyl” intends an alkenyl group of two to six carbon atoms, preferably two to four carbon atoms.

“Substituted alkenyl” refers to alkenyl substituted with one or more substituent groups, and the terms “heteroatom-containing alkenyl” and “heteroalkenyl” refer to alkenyl in which at least one carbon atom is replaced with a heteroatom.

“Aryl” refers to a substituted or unsubstituted monovalent aromatic radical having a single ring (e.g., phenyl) or multiple condensed rings (e.g., naphthyl). Other examples include heterocyclic aromatic ring groups having one or more nitrogen, oxygen, or sulfur atoms in the ring, such as imidazolyl, furyl, pyrrolyl, pyridyl, thienyl and indolyl, among others. Therefore, “aryl” as used herein includes “heteroaryls” having a mono- or polycyclic ring system which contains 1 to 15 carbon atoms and 1 to 4 heteroatoms, and in which at least one ring of the ring system is aromatic. Heteroatoms are sulfur, nitrogen or oxygen.

“Substituted aryl” refers to an aryl as just described that contains one or more functional groups such as lower alkyl, acyl, aryl, halogen, alkylhalos (e.g., CF₃), hydroxy, alkoxy, alkoxyalkyl, amino, alkyl and dialkyl amino, acylarnino, acyloxy, aryloxy, aryloxyalkyl, carboxyalkyl, carboxamido, thio, thioethers, both saturated and unsaturated cyclic hydrocarbons, heterocycles and the like.

“Alkynyl” as used herein refers to a branched or unbranched hydrocarbon group typically although not necessarily containing 2 to about 24 carbon atoms and at least one triple bond, such as ethynyl, n-propynyl, isopropynyl, n-butynyl, isobutynyl, octynyl, decynyl, and the like. Generally, although again not necessarily, alkynyl groups herein contain 2 to about 12 carbon atoms. The term “lower alkynyl” intends an alkynyl group of two to six carbon atoms, preferably three or four carbon atoms. “Substituted alkynyl” refers to alkynyl substituted with one or more substituent groups, and the terms “heteroatom-containing alkynyl” and “heteroalkynyl” refer to alkynyl in which at least one carbon atom is replaced with a heteroatom.

“Alkoxy” as used herein refers to an alkyl group bound through an ether linkage; that is, an “alkoxy” group may be represented as —O—alkyl where alkyl is as defined above. A “lower alkoxy” group intends an alkoxy group containing one to six, more preferably one to four, carbon atoms.

“Allenyl” is used herein in the conventional sense to refer to a molecular segment having the structure —CH═C═CH₂. An “allenyl” group may be unsubstituted or substituted with one or more non-hydrogen substituents.

“Anomer” as used herein means one of a pair of isomers of a cyclic carbohydrate resulting from creation of a new point of symmetry when a rearrangement of atoms occurs at an aldehyde or ketone position.

“Chelerythrine analog” means a compound of formulae (I)-(IV) as defined previously.

“Halo” and “halogen” are used in the conventional sense to refer to a chloro, bromo, fluoro or iodo substituent. The terms “haloalkyl,” “haloalkenyl” or “haloalkynyl” (or “halogenated alkyl,” “halogenated alkenyl,” or “halogenated alkynyl”) refers to an alkyl, alkenyl or alkynyl group, respectively, in which at least one of the hydrogen atoms in the group has been replaced with a halogen atom.

“Heterocycle” or “heterocyclic” refers to a carbocylic ring wherein one or more carbon atoms have been replaced with one or more heteroatoms such as nitrogen, oxygen or sulfur. A substitutable nitrogen on an aromatic or non-aromatic heterocyclic ring may be optionally substituted. The heteroatoms N or S may also exist in oxidized form such as NO, SO and SO₂. Examples of heterocycles include, but are not limited to, piperidine, pyrrolidine, morpholine, thiomorpholine, piperazine, tetrahydrofuran, tetrahydropyran, 2-pyrrolidinone, δ-velerolactam, δ-velerolactone and 2-ketopiperazine, among numerous others.

“Heteroatom-containing” refers to a molecule or molecular fragment in which one or more carbon atoms is replaced with an atom other carbon, e.g., nitrogen, oxygen, sulfur, phosphorus or silicon. “Substituted heterocycle” refers to a heterocycle as just described that contains one or more functional groups such as lower alkyl, acyl, aryl, cyano, halogen, hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like. In other instances where the term “substituted” is used, the substituents which fall under this definition may be readily gleaned from the other definitions of substituents which are presented in the specification as well the circumstances under which such substituents occur in a given chemical compound. One having ordinary skill in the art will recognize that the maximum number of heteroatoms in a stable, chemically feasible heterocyclic ring, whether it is aromatic or non-aromatic, is determined by the size of the ring, degree of unsaturation, and valence of the heteroatoms. In general, a heterocyclic ring may have one to four heteroatoms so long as the heterocyclic ring is chemically feasible and stable.

“Isostere” refers to compounds that have substantially similar physical properties as a result of having substantially similar electron arrangements.

“Substituted”, as in “substituted alkyl” or “substituted alkenyl”, means that in the hydrocarbyl, hydrocarbylene, alkyl, alkenyl or other moiety, at least one hydrogen atom bound to a carbon atom is replaced with one or more substituents that are functional groups such as hydroxyl, alkoxy, thio, amino, halo, silyl, and the like. When the term “substituted” appears prior to a list of possible substituted groups, it is intended that the term apply to every member of that group.

“Effective amount” refers to the amount of a selected compound, intermediate or reactant which is used to produce an intended result. The precise amount of a compound, intermediate or reactant used will vary depending upon the particular compound selected and its intended use, the age and weight of the subject, route of administration, and so forth, but may be easily determined by routine experimentation. In the case of the treatment of a condition or disease state, an effective amount is that amount which is used to effectively treat the particular condition or disease state. Therefore, “effective amount” includes amounts of chelerythrine or chelerythrine analogs that are effective in treating CNS disorders that include, but are not limited to, bipolar disorder, major depressive disorder, schizophrenia, post-traumatic stress disorder, anxiety disorders, attention deficit hyperactivity disorder, and Alzheimer's Disease (behavioral symptoms).

“Anxiety disorders” include affective disorders such as all types of depression, bipolar disorder, cyclothymia, and dysthymia, anxiety disorders such as generalized anxiety disorder, panic, phobias and obsessive-compulsive disorder, stress disorders including post-traumatic stress disorder, stress-induced psychotic episodes, psychosocial dwarfism, stress headaches, and stress-related sleep disorders, and can include drug addiction or drug dependence.

The present invention includes the compositions comprising the pharmaceutically acceptable acid addition salts of compounds of chelerythrine or chelerythrine analogs. The acids which are used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts.

The invention also includes compositions comprising base addition salts of chelerythrine or chelerythrine analogs. The chemical bases that may be used as reagents to prepare pharmaceutically acceptable base salts of chelerythrine analogs that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines.

The compounds of this invention include all stereoisomers (i.e, cis and trans isomers) and all optical isomers of chelerythrine or chelerythrine analogs (eg., R and S enantiomers), as well as racemic, diastereomeric and other mixtures of such isomers, as well as all polymorphs of the compounds.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques. Preferably, the compositions are administered orally, intraperitoneally, or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-otyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of chelerythrine or chelerythrine analog in a pharmaceutical composition of the instant invention that may be combined with the carrier materials to produce a single dosage form will vary depending upon the host treated, the particular mode of administration. Preferably, the compositions should be formulated to contain between about 10 milligrams to about 500 milligrams of active ingredient.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Chelerythrine and Chelerythrine Analogs.

The benzophenanthridine alkaloid chelerythrine (1,2-dimethoxy- 12-methyl[1,3]benzodioxolo[5,6-c]phenanthridinium; C₂₁H₁₈NO₄), also known as toddaline, is extractable either in pure form or as a mixture with other benzophenanthridine alkaloids from Chelidonium majus L., Zanthoxylum simulans, Sanguinaria candensis (or bloodroot), Macleaya cordata, Carydali sevctocozii, Carydali ledebouni, Chelidonium majusm and other members of Papaveracaceae. The major alkaloid in Zanthoxylum simulans, is chelerythrine with smaller quantities of dihydro- and oxy-chelerythrine, N-acetylanomine, skimmianine, fagarine, sitosterol and sesamine.

Representative chelerythrine analogs of the present invention can be synthesized in accordance with the general synthetic methods described below and are illustrated more particularly in the schemes that follow. Since the schemes are illustrative, the invention should not be construed as being limited by the chemical reactions and conditions expressed. The preparation of the various starting materials used in the schemes is well within the skill of persons versed in the art.

Unless specified to the contrary, reactions herein occur at approximately atmospheric pressure and at a temperature of between about 0° C. and the boiling point of any organic solvent used in the reaction. Inert organic solvents such as dichloromethane, diethyl ether, dimethylformamide, chloroform or tetrahydrofuran are preferred solvents in the reactions disclosed herein. Reaction times can range from about one hour to about forty-eight hours, and reactants optionally are stirred, shaken, or agitated. Reactions can be done in one pot or in steps, unless specified to the contrary.

In a purely illustrative example, derivation of commercially available isoquinoline analogs may readily form the third and fourth ring structure (and even the fifth, 1,3-dioxolane ring structure, where applicable) with an appropriately derivitized benzaldehyde or other condensable intermediate, which can be condensed onto the appropriately substituted isoquinoline analog to form chelerythrine or a chelerythrine analog. Alternatively, an electrophilic benzaldehyde may be condensed with an amine (aniline or 1-aminonaphthylene analog) to produce the chelerythrine ring structure containing the 1,3-dioxolane moiety, or alternatively. A 1,2-hydroxy phenolic group may be condensed with dibromomethane or other electrophilic compound to introduce the methylene bridge between the two hydroxyl groups of the hydroxyphenol to produce the 1,3-dioxoane moiety of compounds according to the present invention. Synthetic methods for producing compounds according to the present invention are well known in the art and can be found, for example, in March, Jerry, Advanced Organic Chemistry, 2^{nd} Edition, McGraw-Hill Publishing Company, among numerous others.

PKC Activation and Prefrontal Cortical Function.

The influence of PKC activation on prefrontal cortical function was tested in rats and monkeys performing spatial working memory tasks that are critically dependant upon the integrity of the prefrontal cortex. All procedures were approved by the Yale Institutional Animal Care and Use Committee. Rats were trained on the spatial delayed alternation task in a T maze, or on a control task, spatial discrimination, which has similar motor and motivational demands but depends upon the posterior cortex and not the prefrontal cortex. Performance on the delayed alternation task is dependant upon the length of the delay between trials. The delay was raised as needed to maintain each individual animals performance at approximately 70% correct, allowing room for improvement or impairment in performance following drug administration.

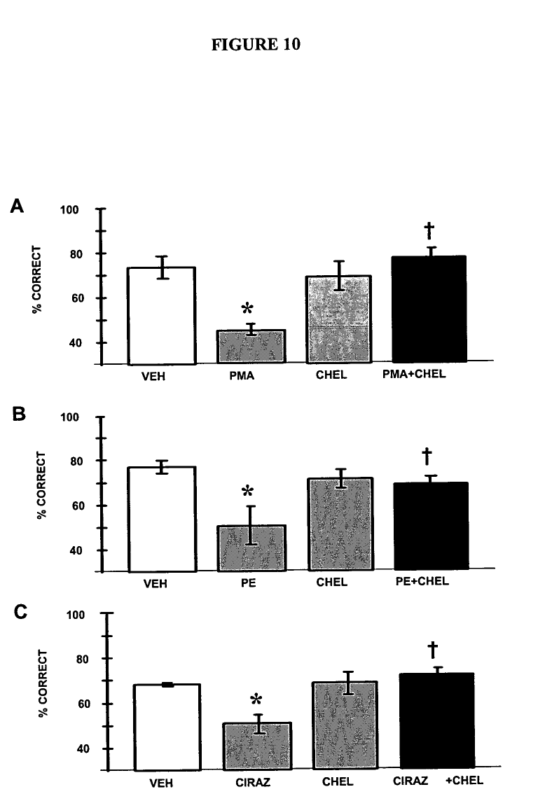

Following behavioral training, rats were implanted with guide cannulae to allow drug infusions into the prefrontal cortex (stereotaxic coordinates from bregma and skull surface: anterior +3.2 mm, lateral ±0.75 mm, ventral −1.7 mm). The infusion needles projected 2.8 mm below the guide cannula such that the infusion site was −4.5 mm ventral to skull surface. Rats were allowed to recover for one week following surgery. Drug treatments were administered only after the animal achieved stable performance (60-80% correct) for two consecutive days. PKC was activated directly using phorbol 12-myristate 13-acetate (PMA), and selectively inhibited with chelerythrine. Local infusion of PMA into the prefrontal cortex in rats (10 min prior to cognitive testing) significantly impaired working memory (FIG. 10A). The PMA-induced working memory impairment was blocked by co-administration of chelerythrine, which had no effect on performance when administered alone (FIG. 10A). In contrast, PMA (5 picograms PMA/0.5 μl) had no effect on performance of the control spatial discrimination task (10 sec delays, mean performance after vehicle: 92.0%±11.0%, mean performance after PMA: 88.0%±13.0%, p=0.587, Tdep test). The absence of PMA effects on the control task demonstrate that the impairment of delayed alternation performance was not due to non-selective motor or motivational effects of the drug treatment, which would be expected to alter both tasks. Instead, the results indicate that PKC activation markedly impairs the cognitive abilities of the prefrontal cortex.

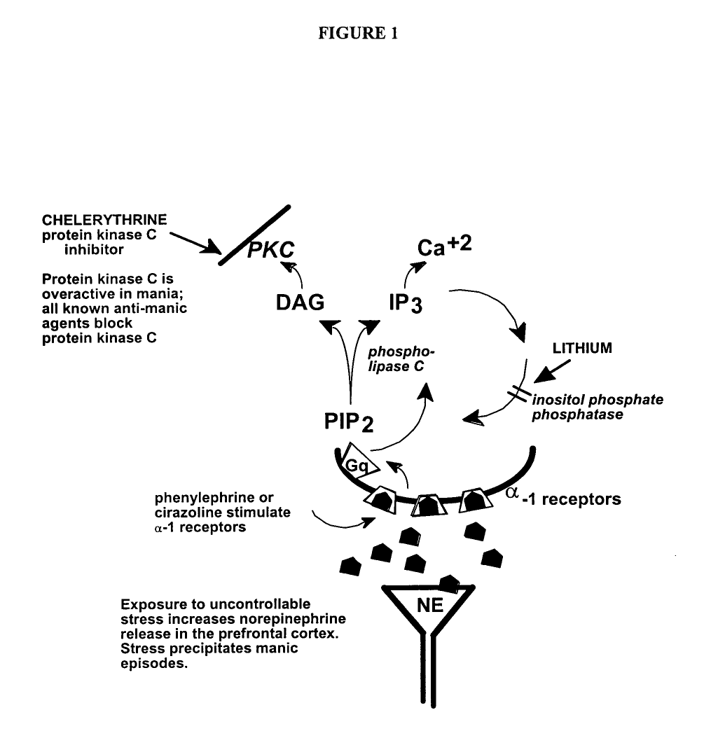

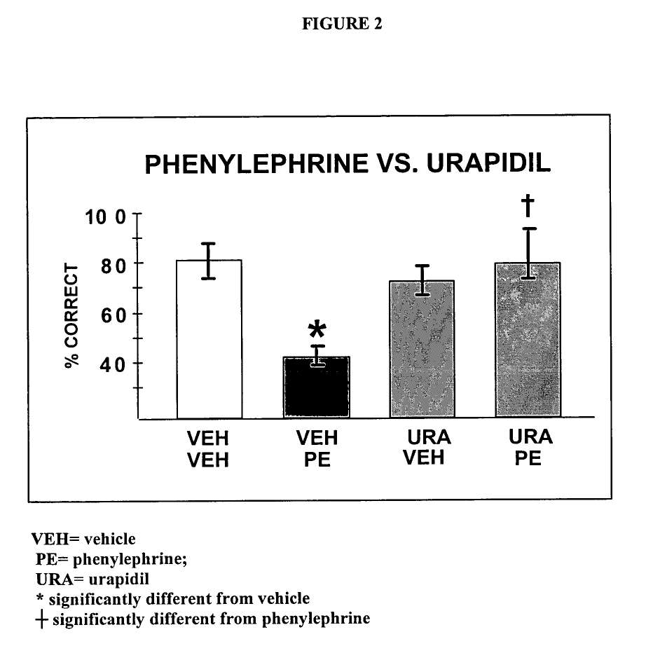

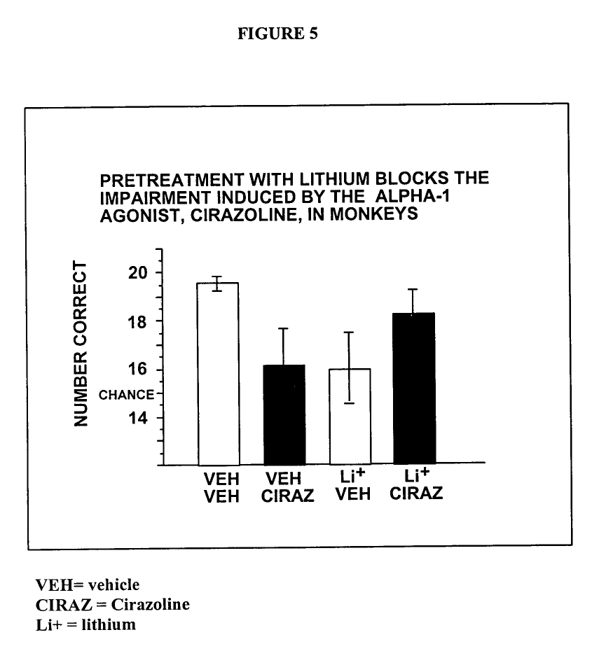

NE α-1 adrenergic receptors are positively coupled to PKC through a Gq-protein linked to the PI intracellular signaling pathway (FIG. 1). Previous studies have shown that infusion of the α-1 adrenergic receptor agonist, phenylephrine, into the prefrontal cortex impairs working memory in both rats (Arnsten et al., 1999; FIGS. 2, 6 and 10B) and monkeys (Mao et al, 1999). Likewise, systemic injections of cirazoline, an α-1 adrenergic receptor agonist that crosses the blood brain barrier, impairs working memory in monkeys (FIGS. 5 and 10C). Thus, PKC was activated indirectly by infusing phenylephrine into the prefrontal cortex in rats (5 min prior to cognitive testing), or by systemic administration of cirazoline in monkeys (intramuscular injections, 30 min prior to cognitive testing). Monkeys had been previously trained on the spatial delayed response task, the task most commonly used to assess prefrontal cortical function in nonhuman primates. The PKC inhibitor, chelerythrine, was administered directly into the prefrontal cortex in rats (5 min prior to cognitive testing), or systemically in monkeys (oral administration, 60 min prior to cognitive testing).

As observed previously, α-1 adrenergic receptor agonists significantly impaired cognitive performance in both rats and monkeys (FIGS. 10B and 10C). This impairment was blocked by the PKC inhibitor, chelerythrine (FIGS. 10B and 10C), indicating that NE α-1 adrenergic receptor stimulation impairs working memory via activation of the PI/PKC intracellular signaling cascade. These findings are particularly meaningful given the previous association between increased levels of NE and mania. Together, these data demonstrate that either direct activation of PKC with a phorbol ester, or indirect activation of PKC through α-1 adrenergic receptor stimulation, is sufficient to impair prefrontal cortical function.

Blocking the Detrimental Effects of Stress.

It has been observed that exposure to stress can precipitate the onset of manic episodes as well as exacerbate the severity of the symptoms. Furthermore, exposure to environmental or pharmacological (FG7142) stressors impairs cognitive performance on tasks dependent on the prefrontal cortex, while having no effects on control, non-prefrontal cortically dependent tasks in both humans and research animals. During stress, NE-containing cells fire rapidly in a “tonic” mode, releasing high levels of NE throughout the brain, including the prefrontal cortex. This “tonic” mode is associated with poor cognitive performance and distractibility, which is likely caused by high levels of NE release stimulating α-1 adrenergic receptors in the prefrontal cortex.

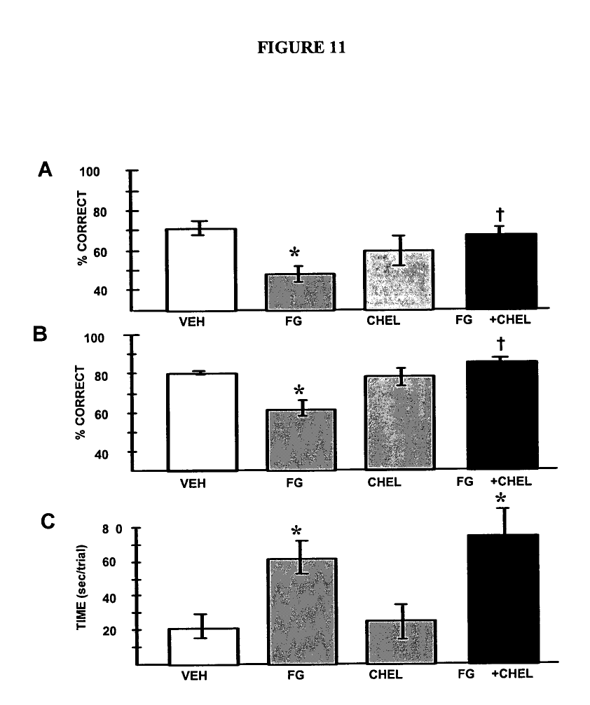

The anxiogenic stressor, FG7142, was administered systemically to rats (interperitonial injection) or monkeys (intramuscular injection) 30 min prior to cognitive testing. Chelerythrine was administered directly into the prefrontal cortex in rats (15 min prior to cognitive testing), or systemically in monkeys (oral administration, 60 min prior to cognitive testing). As observed previously, FG7142 significantly impaired working memory in both rats and monkeys (FIGS. 11A and 11B). This cognitive impairment was blocked by chelerythrine (FIGS. 11A and 11B), consistent with stress-induced activation of PKC.

It is important to note that cortical infusions of chelerythrine in rats did not reverse other aspects of the stress response that are unrelated to prefrontal cortical function. For example, stressors such as FG7142 induce freezing behaviors in rodents that effectively lengthen the delays and increase the memory demands of the task (FIG. 11C). Remarkably, infusions of chelerythrine into the rat prefrontal cortex restored normal cognitive performance even though they had no effect on response time in stressed animals (FIG. 11C). These findings emphasize that endogenous (stress) as well as exogenous (PMA) activation of PKC signaling has marked, detrimental effects on prefrontal cortical function, suggesting that stress exposure precipitates manic episodes by increasing PKC activity.

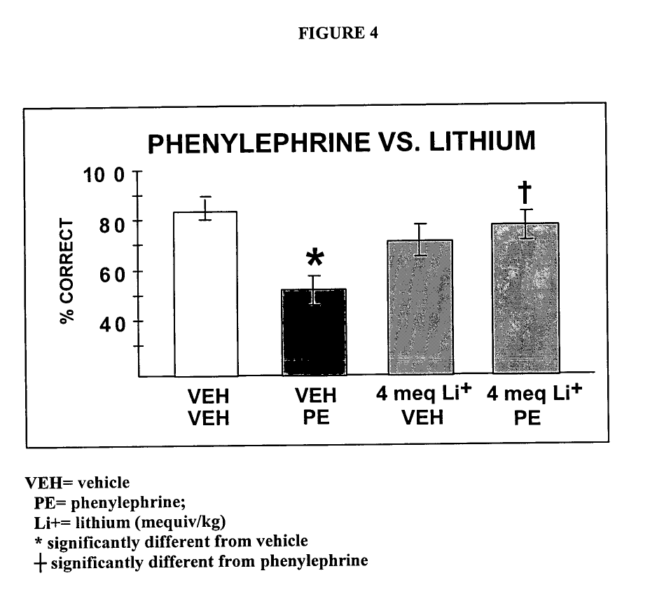

The impairment induced by alpha-1 adrenergic agonist infusion into the prefrontal cortex is reversed by a dose regimen of lithium treatment known to suppress phosphotidyl inositol turnover in rats (Arnsten et al., 1999; FIG. 4). Applicants have determined that in monkeys, a lithium dose range used to treat manic patients (5-7.5 mg/kg p.o.) can reverse the deficits induced by systemic administration of the alpha-1 agonist, cirazoline (FIG. 5).

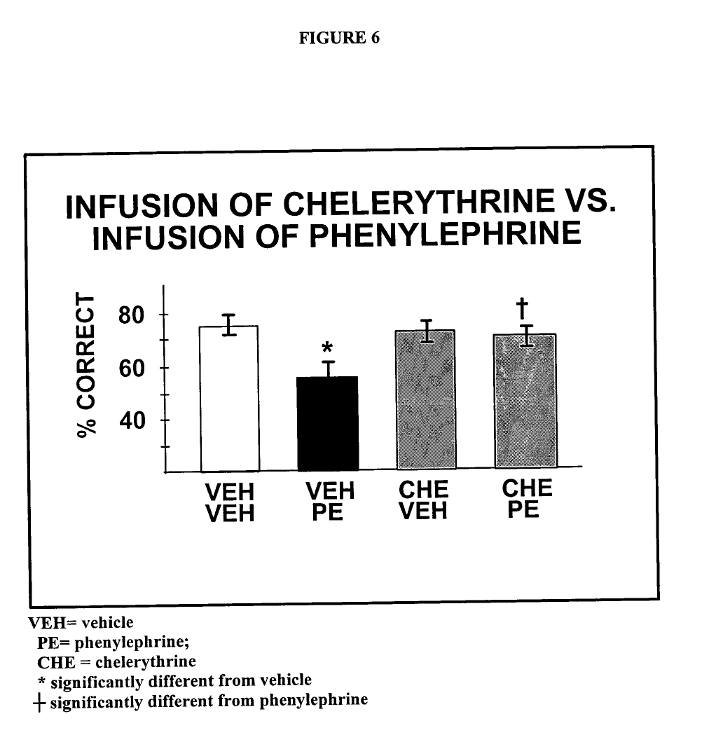

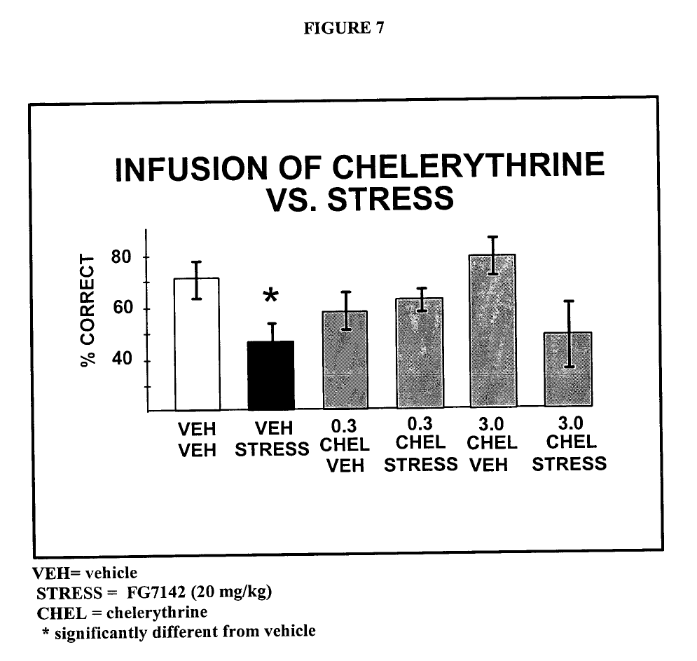

The effects of small quantities of chelerythrine infused directly into the prefrontal cortex in rats were examined. Infusions of chelerythrine (0.3 μg/0.5 μl) into the PFC had no effect on performance by themselves, but significantly reversed the detrimental effects of either an alpha-1 agonist (FIGS. 6 and 10B) or stress exposure (FIGS. 7 and 11A). Interestingly, infusion of a higher dose of chelerythrine (3.0 μg/0.51 μl) did not reverse the stress response, even though this dose had no effect when infused by itself. These data indicate that there is a defined dose range for beneficial drug effects, unrelated to observable side effects. These findings strongly supported the hypothesis that stress-induced prefrontal cortical cognitive impairment involves activation of protein kinase C in the prefrontal cortex.

The invention is described further in the following examples, which are illustrative and in no way limiting.

### EXAMPLE 1

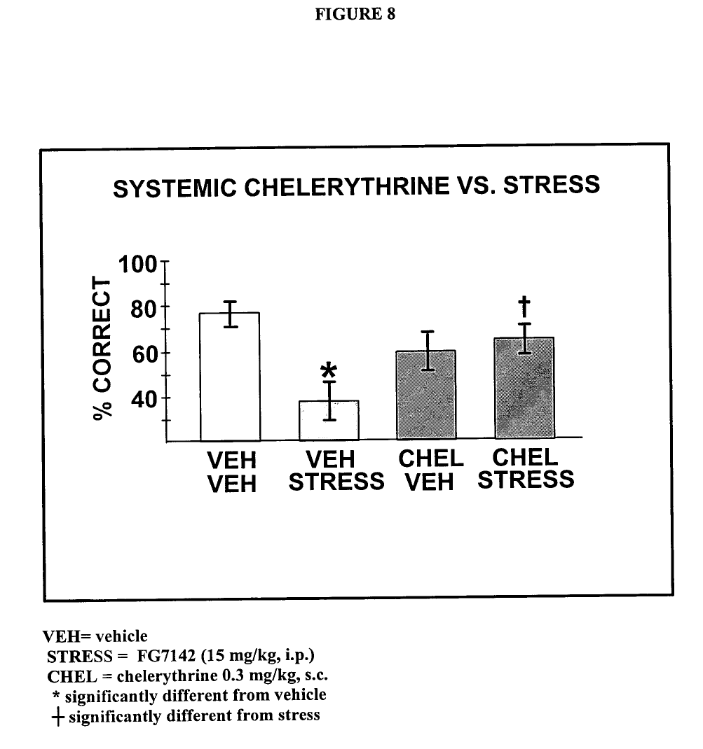

Rats were injected with 0, 0.3, or 3.0 mg/kg chelerythrine s.c. in water approximately 45 minutes before cognitive testing; they receive an injection of the pharmacological stressor, FG7142 (15 mg/kg, i.p.) or vehicle 30 minutes prior to cognitive testing. All drug treatments occur at least one week apart, and the order of treatments is counterbalanced between animals. As illustrated in FIG. 8, injection of the lower dose of chelerythrine (0.3 mg/kg, s.c., 45 min) significantly reversed the detrimental effects of stress exposure (p=0.018, n=4). The higher dose of chelerythrine (3.0 mg/kg) did not reverse the cognitive deficits due to stress, although it had no effect on behavior on its own (average of 72.5% correct, similar to vehicle). Careful behavioral observations in the home cage and during cognitive testing indicated no significant side effects with chelerythrine administration by itself at either dose; occasionally animals were reported to be “a little slower but normal”. All ratings were performed by experimenters who were very familiar with the normative behavior of the animal but were unaware of the drug treatment conditions.

### EXAMPLE 2

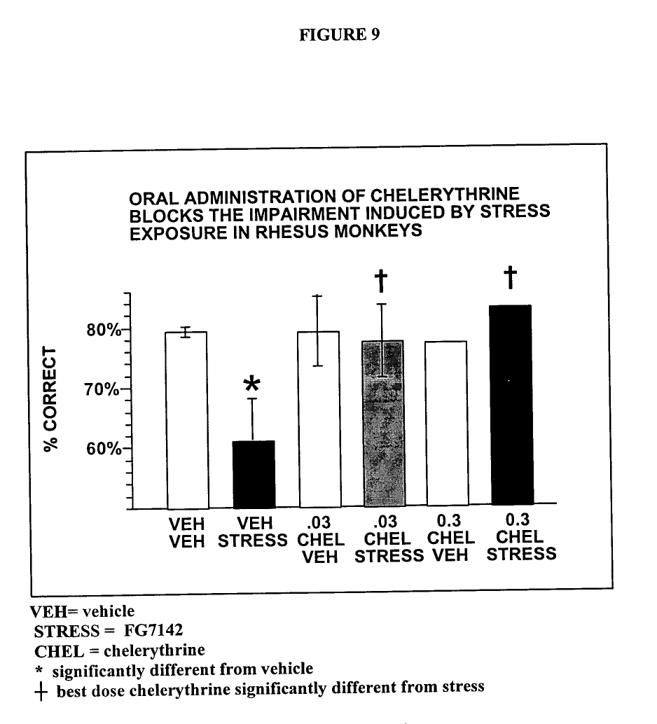

Chelerythrine was administered orally to rhesus monkeys at doses of either 0.03/kg or 0.3 mg/kg 60 min before cognitive testing, 30 minutes prior to stress exposure (FG7142 0.2-1.0 mg/kg, i.m.). In addition to cognitive testing, monkeys are also assessed for changes in sedation, agitation, aggression, motivation, food intake, and both fine and gross motor abilities. Four monkeys were tested. Chelerythrine pretreatment significantly reversed the detrimental effects of stress on prefrontal cortical function (FIG. 9; p<0.05, n=4). Half of the monkeys showed complete protection with the 0.03 mg/kg dosage; the other half required 0.3 mg/kg for full reversal. Chelerythrine by itself had no effect on cognitive performance, and was well-tolerated with no side effects at either dose. Combined dose data are shown in FIG. 11.

It is to be understood by those skilled in the art that the foregoing description and examples are illustrative of practicing the present invention, but are in no way limiting. Variations of the detail presented herein may be made without departing from the spirit and scope of the present invention as defined by the following claims.

1. A method for treating a CNS disorder or impaired cognitive performance in a subject comprising administering to said subject in need thereof an effective amount of a pharmaceutical composition comprising a compound or a stereoisomer, pharmaceutically acceptable salt, solvate or polymorph thereof according to the structure: 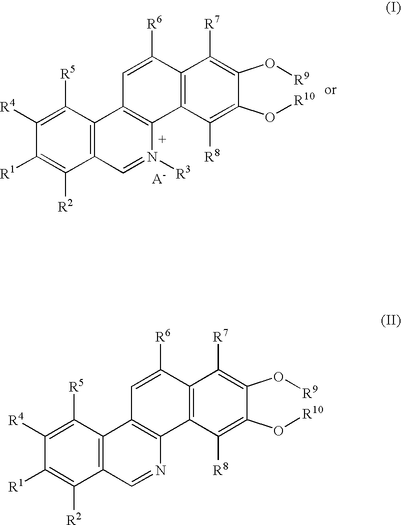
wherein:
R¹ and R² are independently selected from H, C₁-C₃ alkyl, F, Cl, Br, I, OH, O(C₁-C₆ alkyl), O—C(═O)—(C₁-C₆)alkyl or C(═O)—O—(C₁-C₆)alkyl;
R³ is H or a C₁-C₆ alkyl group;
R⁴, R⁵, R⁶, R⁷ and R⁸ are independently selected from H, C₁-C₆ alkyl, F, Cl, Br, I, OH, —(CH₂)ₙO(C₁-C₆ alkyl), —(CH₂)ₙO—C(═O)—(C₁-C₆)alkyl or —(CH₂)ₙC(═O)—O—(C₁-C₆)alkyl;
R⁹ and R¹⁰ are independently H, C₁-C₆ alkyl or together form a —(CH₂)ₘ— group to produce a 5-7-membered ring;
n is from 0 to 5;
m is from 1 to 3;
and A- is a pharmaceutically acceptable anion of a pharmaceutical salt, which forms a salt with the quaternized amine group, optionally in combination with a pharmaceutically acceptable carrier additive or excipient.

2. A method of claim 1 wherein R¹ and R² are both OCH₃ groups, R³ is a CH₃ group, R⁴, R⁵, R⁶, R⁷ and R⁸ are each H, R⁹ and R¹⁰ are each H, CH₃ or together form a —CH₂— group to produce a five-membered ring;
and A⁻ is Cl—, citrate or phosphate.

3. A method of claim 2, wherein R⁹ and R¹⁰ together form a —CH₂— group to produce a five-membered ring.

4. A method of claim 1, wherein the CNS disorder is a bipolar disorder.

5. A method of claim 1, wherein the CNS disorder is an anxiety disorder.

6. A method of claim 1, wherein the CNS disorder is stress-induced.

7. A method of claim 1, wherein the CNS disorder is attention deficit hyperactivity disorder (ADHD).

8. A method of claim 1, wherein the CNS disorder is schizophrenia.

9. A method of claim 1, wherein said subject is treated for impaired cognitive performance.

10. A method of claim 1, wherein the CNS disorder is associated with enhanced PKC activity.

11. A method of claim 1, wherein the impaired cognitive performance is induced or exacerbated by stress.

12. A method of claim 1, wherein the pharmaceutical composition is administered orally and the subject is a human.

13. A method of claim 9, wherein the pharmaceutical composition is administered orally and the subject is a human.

14. A method of claim 10, wherein the pharmaceutical composition is administered orally and the subject is a human.

15. A pharmaceutical composition comprising an effective amount of a compound or a stereoisomer, pharmaceutically acceptable salt, solvate or polymorph thereof according to the structure: 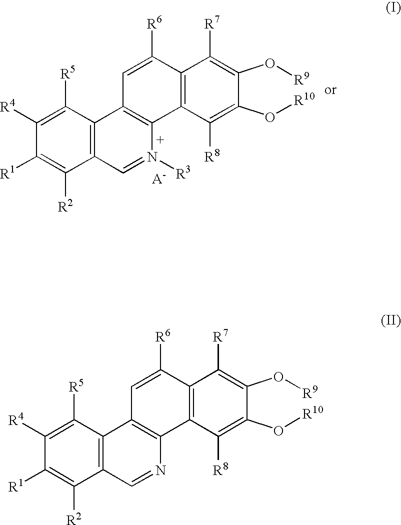
wherein:
R¹ and R² are independently selected from H, C₁-C₃ alkyl, F, Cl, Br, I, OH, O(C₁-C₆ alkyl), O—C(═O)—(C₁-C₆)alkyl or C(═O)—O—(C₁-C₆)alkyl;
R³ is H or a C₁-C₆ alkyl group;
R⁴, R⁵, R⁶, R⁷ and R⁸ are independently selected from H, C₁-C₆ alkyl, F, Cl, Br, I, OH, —(CH₂)ₙO(C₁-C₆ alkyl), —(CH₂)ₙO—C(═O)—(C₁-C₆)alkyl or —(CH₂)ₙC(═O)—O—(C₁-C₆)alkyl;
R⁹ and R¹⁰ are independently H, C₁-C₆ alkyl or together form a —(CH₂)ₘ— group to produce a 5-7-membered ring;
n is from 0 to 5;
m is from 1 to 3;
and A- is a pharmaceutically acceptable anion of a pharmaceutical salt, which forms a salt with the quaternized amine group, in combination with a pharmaceutically acceptable carrier, additive or excipient.

16. A composition of claim 14 wherein R¹ and R² are both OCH₃ groups, R³ is a CH₃ group, R⁴, R⁵, R⁶, R⁷ and R⁸ are each H, R⁹ and R¹⁰ are each H, CH₃ or together form a —CH₂— group to produce a five-membered ring;
and A⁻ is Cl—, citrate or phosphate.

17. A composition of claim 15, wherein R⁹ and R¹⁰ together form a —CH₂— group to produce a five-membered ring.

18. A method of treatment comprising administering to a subject suffering from manic episodes associated with enhanced PKC activity a therapeutically effective amount of a composition according to claim 15.

19. A method of claim 18, wherein the manic episodes are also stress induced.

20. A method comprising protecting a subject from developing a CNS disorder by administering to the subject a therapeutically effective amount of a pharmaceutical composition according to claim 15.
